(19) United States
(12) Patent Application Publication
(10) Pub. No.: US 2025/0297314 A1
(43) Pub. Date: Sep. 25, 2025

(54) DETERMINING THE RISK OF DEATH OF A SUBJECT INFECTED WITH A RESPIRATORY VIRUS BY MEASURING THE EXPRESSION LEVEL OF THE CD74 GENE

(71) Applicants: bioMérieux, Marcy-l'Étoile (FR); Hospices Civils de Lyon, Lyon (FR); Universite Claude Bernard Lyon 1, Villeurbanne (FR)

(72) Inventors: Claire TARDIVEAU, Saint Germain au Mont d'Or (FR); Anne-Claire LUKASZEWICZ, Lyon (FR); Guillaume MONNERET, Lyon (FR); Fabienne VENET, Lyon (FR)

(21) Appl. No.: 18/880,056

(22) PCT Filed: Jul. 5, 2023
(86) PCT No.: PCT/EP2023/068512

§ 371 Date: Dec. 30, 2024

(30) Foreign Application Priority Data
Jul. 6, 2022 (EP) 22183393.2
Jul. 6, 2022 (EP) 22183395.7

(51) Int. Cl. C12Q 1/6883

(52) U.S. Cl. CPC C12Q 1/6883; C12Q 2600/118; C12Q 2600/158

(57) ABSTRACT

The invention relates to an in vitro or ex vivo method for determining the risk of death in a subject infected with a respiratory virus, for example SARS-CoV-2, comprising a measurement, in a biological sample of said subject, of the level of expression of the CD74 gene; the invention also relates to associated kits.

### TECHNICAL FIELD

The present invention relates to the technical field of methods and kits for in vitro diagnostics. In particular, the invention relates to methods and kits that make it possible to determine the risk of death of a subject infected with a respiratory virus, particularly with a respiratory virus such as SARS-CoV-2 or a variant thereof.

### PRIOR ART

To date, the coronavirus (COVID-19) pandemic caused by the severe acute respiratory syndrome coronavirus 2 (SARS-CoV-2) has infected more than 550 million patients globally and caused more than 6.3 million deaths.

The severity of the disease varies considerably from one patient to another, and the majority of patients are asymptomatic or exhibit minimal symptoms such as fever, a cough and/or shortness of breath. Nevertheless, 5 to 10% of patients require intensive care due to the rapid evolution (9 to 12 days) to a more severe or critical form with the development, for example, of acute respiratory distress syndrome (ARDS) and/or severe hypoxemia, acute pulmonary lesions (APL), multiple organ failure, and even death (C. Huang et al., 2020).

Immune response is of critical importance in the physiopathology of COVID-19, and the most severe phenotype of infected patients on admission to intensive care is characterized by a complex immune profile which evolves overtime (E. Z. Ong et al., 2020).

In spite of everything, in patients suffering from severe COVID-19, the immune response can be defined by altered inflammatory and immune responses with pronounced lymphopenia, a high neutrophil and monocyte count, a decrease in monocyte HLA-DR expression, a moderate plasma cytokine storm, inadequate type-I interferon signaling response and down-regulation of IFN-stimulated genes (ISG) (F. Venet et al., 2021).

Over recent months, several research groups have attempted to identify the risk factors characteristic of severe evolution of the COVID-19 disease, in order in particular to identify patients who are at high risk of developing said severe forms.

For example, in a high-dimensional study combining some fifty clinical characteristics and around two hundred immunological characteristics, three distinct immunotypes associated with disease severity were described (Mathew et al., 2020).

In the context of a vast immune evaluation study combining cellular data obtained by flow cytometry, soluble immune markers (multiplex cytokine analysis), RNA expression (Nanostring) and serology (ELISA), a core peripheral blood immune signature was discovered in patients suffering from COVID-19, with this signature potentially making it possible to identify immunopathology parameters, correlate with disease severity and also anticipate clinical progression (Laing et al., 2020).

More recently, it has also been established that the longitudinal trajectories of 11 circulating immunity-based biomarkers could be associated with patients' mortality when they were increased (10) or decreased (1), thereby providing initial evidence that immunity-based biomarkers could make it possible to obtain an early warning of the outcome for patients suffering from COVID-19 (Abers et al., 2021). Gene expression profiles have also been described for predicting the outcome for patients suffering from COVID-19 (Guardela B et al., 2021).

In this context, the biomarker CD177 has been described as possibly being associated with severity and the risk of death of patients suffering from COVID-19. Particularly, the stability of CD177 protein levels in serum from patients suffering from severe COVID-19 over the course of the disease is described as a sign of a less favourable prognosis, possibly leading to death (Lévy Y et al., 2021).

Nevertheless, in light of the global prevalence, there is still a need to identify new biomarkers that offer other alternatives for effectively predicting the risk of death of subjects infected with respiratory viruses, particularly the respiratory viruses responsible for COVID-19, in order to be able to adapt treatment, preferentially early on, through guided therapies, and thereby improve those subjects' chances of survival.

### SUMMARY OF THE INVENTION

A first subject of the invention relates to an in vitro or ex vivo method for determining the risk of death of a subject infected with a respiratory virus, comprising a step of measuring the expression level of the CD74 gene, and optionally the expression of at least one other additional gene selected from TDRD9, IL1R2 and/or CD177, in a biological sample from said subject.

Advantageously, the method according to the invention may also further comprise measuring the expression of one or more additional genes involved in host response, such as those listed in table 2.

The method according to the invention thus entirely advantageously makes it possible to determine the risk of death of a subject infected with a respiratory virus, particularly with a respiratory virus such as SARS-CoV-2 or a variant thereof.

The conclusion regarding the risk of death is particularly drawn by comparing the CD74 expression level to that of a reference value which may correspond to the average CD74 expression level obtained from biological samples originating from a population of subjects who are not infected with any respiratory virus, or to the average CD74 expression level obtained from biological samples originating from a population of subjects who are infected with a respiratory virus and who are known to have survived following infection, particularly in the 28 days following infection.

In particular, on the basis of the result of the comparison of the CD74 expression level to a reference value, a conclusion regarding an increased risk of death of the subject is drawn when a decrease in expression is identified.

Advantageously, the method according to the invention comprises determining the expression of the CD74 gene and of the additional gene, IL1R2. Thus, the method according to the invention comprises the following steps of:
measuring the expression level of the CD74 gene and of the additional gene IL1R2,
comparing the expression level of said genes for said biological sample, or a value derived from this quantity, to a predetermined threshold value corresponding to the average expression level of said genes obtained from biological samples originating from a population of subjects who are not infected with any respiratory virus, or to the average expression level of said obtained from biological samples originating from a population of subjects who are infected with a respiratory virus and who are known to have survived following infection, particularly in the 28 days following infection, and
drawing a conclusion regarding the risk of death on the basis of the result of the comparison.

The method thus makes it possible to conclude that there is an increased risk of death of the subject when under-expression of CD74 and over-expression of IL1R2 is demonstrated in the biological sample to be tested.

Advantageously, the method according to the invention comprises determining the expression of the CD74 gene and of the additional genes, IL1R2 and CD177. Thus, the method according to the invention comprises the following steps of:
measuring the expression level of the CD74 gene and of the additional genes IL1R2 and CD177,
comparing the expression level of said genes for said biological sample, or a value derived from this quantity, to a predetermined threshold value corresponding to the average expression level of said genes obtained from biological samples originating from a population of subjects who are not infected with any respiratory virus, or to the average expression level of said obtained from biological samples originating from a population of subjects who are infected with a respiratory virus and who are known to have survived following infection, particularly in the 28 days following infection, and
drawing a conclusion regarding the risk of death on the basis of the result of the comparison.

The method thus makes it possible to conclude that there is an increased risk of death of the subject when under-expression of CD74 and over-expression of IL1R2 and CD177 is demonstrated in the biological sample to be tested.

Another subject of the invention relates to a kit for the in vitro or ex vivo measurement of the expression of CD74 in a biological sample, comprising means for determining the CD74 expression level in said sample. The kit may also comprise means for determining the expression level of at least one other additional gene selected from TDRD9, IL1R2 and CD177 and means for determining at least one gene involved in host response, such as those listed in table 2.

Advantageously, the kit may particularly comprise a negative control sample calibrated to contain the quantity of CD74 that corresponds to the quantity or concentration representative of the expression level measured in a pool of samples from subjects who are not infected with a respiratory virus, and/or a positive control sample calibrated to contain the quantity of CD74 that corresponds to the average quantity measured in a pool of samples from subjects who did not survive following infection with a respiratory virus.

Another subject of the invention relates to the use of the kit for determining the risk of death, preferably the risk of death in the 28 days post-infection, of a subject infected with a respiratory virus such as SARS-CoV-2 or a variant thereof.

### DESCRIPTION OF THE FIGURES

FIG. 1 is a diagram representing the importance of variables in different machine learning models used to predict mortality in 28 days within the RICO sub-cohort. The markers CD74, TDRD9, and ILR2 were identified in the 5 models, and CD74 has good results. CD177 was not captured by the Lasso model but was the fourth marker of interest identified by the cumulative importance of the variable.

FIG. 2 shows violin plots representing the expression of CD74 and of additional genes associated with mortality which were identified by the machine learning models. The p values were calculated using a Wilcoxon test. The left-hand side shows healthy volunteers, the middle shows patients who were alive on the 28th day following admission, and the right-hand side shows patients who died before the 28th day.

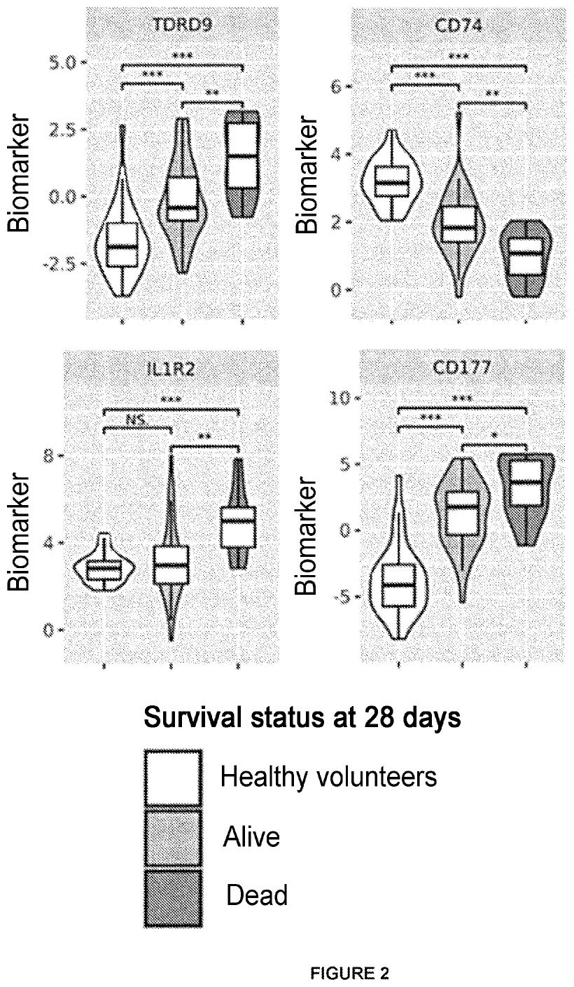

FIG. 3 describes associations between the mRNA levels of CD74, CD177, TDRD9, and IL1R2 measured on admission and the incidence of death in the 28 days following admission. The cumulative events, not adjusted for death, were obtained with Kaplan-Meier estimations. The patients were stratified into 2 groups based on the Youden threshold determined for each mRNA for the prediction of mortality in 28 days in the total population of patients on admission. The p value from the log-rank test is indicated.

### DETAILED DESCRIPTION OF THE INVENTION

Certain terms and expressions used in the context of the invention are detailed hereinbelow.

A first subject of the invention relates to an in vitro or ex vivo method for determining the risk of death of a subject infected with a respiratory virus, comprising a step of measuring the expression level of the CD74 gene in a biological sample from said subject.

Entirely surprisingly, it has been observed that measuring the expression of the CD74 gene made it possible to determine the risk of death of a subject infected with a respiratory virus. Thus, in the context of the increasing importance of personalized medicine, subjects having an increased risk of death could benefit from personalized treatment.

This is all the more surprising since, in the same infected subject, the standard immunological parameters measured on admission to intensive care and used as references by practitioners, namely monocyte HLA-DR and the plasma interleukins IL-6 and IL-10, are not significantly associated with death in the 28 days following admission. This therefore demonstrates the benefit of measuring the expression level of the CD74 gene and of the additional genes according to the invention.

The method which is a subject of the invention has the advantage of being able to easily assess the risk of death of a subject, for example a patient admitted to the resuscitation unit or emergency department, using a directly-measurable biomarker, the measurement of which can be carried out directly in the healthcare facility where the subject has been admitted or in a nearby laboratory. Moreover, the measurement of the biomarker CD74 or of the other additional biomarkers of the invention, is entirely suited to being performed by automated analysis devices or “rapid” tests.

“Biomarker” or “marker” means a biological characteristic which can be objectively measured and which is indicative of normal or pathological biological processes or of a pharmacological response to a therapeutic intervention. For the purposes of the present description, the biomarkers are genes and the expression level thereof can be detected in particular at the transcript level, particularly mRNA transcripts.

The CD74 gene is located on chromosome 5 and encodes the γ chain of MHC II, or Ii (or Iγ) fragment, also referred to as CD74 (“Cluster of Differentiation 74”). This is the invariant fragment Ii, a polypeptide that enables the formation, maturation and transport of MHC II during the formation thereof in antigen-presenting cells. More particularly, CD74 is heavily involved in antigen presentation and CD4+ T cell activation (Cresswell, 1994). It also serves as a cell surface receptor for the macrophage migration inhibitory factor (MIF) cytokine (Farr et al., 2020) which, when linked to the encoded protein, initiates cell survival and proliferation pathways.

The sequence of the CD74 gene can be accessed at the NCBI under the reference NC_000005.10

The expression “risk of death of a subject” refers to the risk of the subject dying in the days following infection with a respiratory virus or in the days following admission to a healthcare facility following infection. In particular, reference is made to an increased risk of death when the subject has a statistically significant risk of death in the days following infection, generally compared to infected subjects who are known to have survived, or uninfected subjects.

Advantageously, the method according to the invention makes it possible to determine a risk of death of a subject in the 28 days following the date on which infection is confirmed, also referred to as days post-infection, particularly using a test for detecting a respiratory virus.

The term “subject” denotes a human, and the subject is preferably a patient. The patient is a person who has been in contact with a healthcare professional, particularly a doctor, or a medical structure or healthcare facility.

According to a particular embodiment, the subject is a patient within a healthcare facility, preferably within a hospital, more preferably within the emergency department, resuscitation department, intensive care unit (ICU) or ongoing care unit, most particularly a patient in the ICU.

“Respiratory virus” means a virus which infects the respiratory tract and/or the lungs. Such viruses are conventionally found in samples taken from the nose, throat and/or mouth of a subject, particularly in nasal samples or nasopharyngeal samples (which require a sample to be taken from more deeply within the nose), oropharyngeal samples (which require a sample to be taken from more deeply in the throat), or saliva.

The expression “subject infected with a respiratory virus” means a subject for whom the test for detecting the presence of a respiratory virus has returned a positive result. These subjects, particularly those who develop more severe forms of the disease, are subjects for whom it is even more relevant to implement the method according to the invention, measuring the CD74 expression level.

Conversely, a subject uninfected with a respiratory virus is a subject for whom the rest for detecting the presence of a respiratory virus is negative.

As examples of respiratory viruses, mention may be made of seasonal coronaviruses, the SARS-CoV-2 virus (“Severe Acute Respiratory Syndrome Coronavirus-2”), regardless of the variants thereof, the flu virus, respiratory syncytial virus (RSV), rhinoviruses, metapneumoviruses, parainfluenza viruses and adenoviruses. To date, the known variants of the SARS-CoV-2 are in particular the variants thereof referred to as British, Brazilian, South African, American, Indian or Omicron. Knowledge of these variants and their names is evolving, and the invention is applicable to all of them (https://www.who.int/en/activities/tracking-SARS-CoV-2-variants/).

The “British variant” of SARS-CoV-2 was discovered on 20th Sep. 2020 in Kent (South-East England). The United Kingdom informed the World Health Organization (WHO) of the circulation of this variant on the 14 December. Initially referred to as VUI 202012/01 (for Variant Under Investigation, year 2020, month 12, variant 01), it was soon renamed, on 18th Dec. 2020, as VOC 202012/01 (for Variant Of Concern), then as Alpha variant. It belongs to the B.1.1.7 lineage in the phylogenetic tree, and comprises the deletion 69-70, also referred to as ΔH69/V70. The Brazilian variant P.1 (Gamma variant) is a descendent of the B.1.1.28 lineage. This Brazilian variant P.1 contains numerous mutations, in particular the mutations E484K, K417T and N501Y. The Japanese variant, which is derived from a lineage present in Brazil (B.1.1.28), contains a very high number of genetic alterations. It comprises twelve amino acid mutations in the spike protein, particularly the mutations N501Y, E484K and K417T. The South African variant (Beta variant), referred to as 501Y.V2 and belonging to the lineage B.1.351, also contains various mutations including three, K417N, E484K and N501Y, which are located in the RBD domain, the receptor binding domain, of the spike protein. The Indian variant, referred to as the Delta variant, belongs to the lineage B.1.617.2 and contains the mutations S417N and S484K. Finally, there is also the Omicron variant, identified in November 2021 and belonging to the lineage B.1.1.529.

According to a particular embodiment, the respiratory virus is a coronavirus, in particular SARS-CoV-2 or a variant thereof, for instance the Alpha, Beta, Gamma, Delta or Omicron variants.

The expressions “test for detecting a respiratory virus”, “test for diagnosing a respiratory virus” or “test for detecting the presence of an infection with a respiratory virus” are synonymous and refer to any test known to those skilled in the art making it possible to draw such a conclusion, particularly tests for detecting the DNA or RNA of said respiratory virus, such as PCR tests, antigen tests or self-tests.

“Biological sample” refers here to any sample originating from a subject, which may be of different natures, such as blood or derivatives thereof, sputum, urine, stools, skin, cerebrospinal fluid, bronchoalveolar lavage fluid, abdominal cavity puncture fluid, saliva, gastric secretions, sperm, seminal fluid, tears, spinal cord, trigeminal nerve ganglion, adipose tissue, lymphoid tissue, placental tissue, gastrointestinal tract tissue, genital tract tissue, or central nervous system tissue.

In particular, the biological sample may be a biological fluid, such as a blood sample or a blood-derived sample, which may particularly be chosen from total blood (as collected from a vein, i.e. containing white and red blood cells, platelets and plasma), plasma, serum, and any types of cells extracted from the blood, for instance peripheral blood mononuclear cells (PBMCs, containing B lymphocytes, T lymphocytes, NK cells, dendritic cells and monocytes), subsets of B cells, purified monocytes, or neutrophils.

According to a preferred embodiment, the biological sample implemented in the method according to the invention is a blood sample, preferably a total blood sample.

For the purposes of the present description, the biological sample from a subject, namely from a subject for whom the risk of death is to be determined, corresponds to the biological sample to be tested, or test sample, as opposed to the reference sample used for comparison.

For the purposes of the present invention, the expression “reference value” or “predetermined reference value” is synonymous with the expressions “control value” or “threshold value” and serves as a point of comparison for determining whether the expression level of a target gene is decreased or increased.

According to the method which is a subject of the invention, the risk of death of a subject infected with a respiratory virus is particularly determined by implementing a step of measuring the expression level of the CD74 gene.

Measuring the expression level of a gene is well known to those skilled in the art and consists particularly in quantifying at least one expression product of the gene. For the purposes of the present invention, the expression product of a gene may be any biological molecule resulting from the expression of said gene. More particularly, the expression product of the gene may be a transcript.

“Transcript” means RNA, and in particular messenger RNA (mRNA) resulting from the transcription of the gene. More specifically, the transcripts are RNAs produced by the transcription of a gene followed by post-transcriptional modifications of the pre-RNA forms.

According to a preferred embodiment, the measurement of the expression level of CD74, and optionally that of one or more additional genes as defined below, is carried out at the RNA level, in particular at the messenger RNA (mRNA) level, in a biological sample from a subject infected with a respiratory virus. According to this embodiment, the measurement of the expression level of the CD74 gene therefore relates to determining the level of mRNA of said gene.

As mentioned previously, measuring the expression level of a gene is well known to those skilled in the art. In the case of a transcript, particularly mRNA, the measurement may be performed by a direct method, by any process known to those skilled in the art which makes it possible to determine the presence of said transcript in the biological sample, or by indirect detection of the transcript after conversion of the latter into DNA, or after amplification of said transcript or after amplification of the DNA obtained after conversion of said transcript into DNA. Numerous methods exist for the detection of nucleic acids and are well known to those skilled in the art (see for example Kricka et al., Clinical Chemistry, 1999, no. 45(4), p. 453-458; Relier G. H. et al., DNA Probes, 2nd Ed., Stockton Press, 1993, sections 5 and 6, p. 173-249).

The expression of the genes may particularly be measured by Reverse Transcription-Polymerase Chain Reaction or RT-PCR, preferably by quantitative RT-PCR or RT-qPCR (for example using the FilmArray® technology or the Biomark™ platform from Fluidigm), by sequencing (preferably by high-throughput sequencing) or by hybridization techniques (for example with hybridization microarrays or by techniques of the NanoString® nCounter® type). All of these methods are also well known to those skilled in the art, and it is not necessary to describe them in detail here.

According to a particular embodiment, the CD74 expression is measured using a molecular detection method, particularly by RT-PCR, sequencing or hybridization. The expression is preferably measured by RT-PCR, and particularly by RT-qPCR.

According to a particular embodiment, the CD74 expression level is measured by quantitative RT-qPCR detection of the mRNA transcripts of CD74.

The measurement of the expression level makes it possible to determine the quantity of one or more CD74 transcripts in the biological sample or also to give a value derived therefrom.

Thus, according to a particular embodiment, the expression level of the CD74 gene is a value derived from the quantity of transcripts thereof. By way of example, a value derived from the quantity of transcripts may for example be the absolute concentration, calculated by virtue of a calibration curve obtained from successive dilutions of a solution of amplicons having a given concentration. It may also correspond to the value of the normalized and calibrated quantity, such as the CNRQ (Calibrated Normalized Relative Quantity, (Hellemans et al (2007), Genome biology 8(2):R19), which integrates the values of a reference sample (or of a calibrator) and of one or more housekeeping genes (also referred to as reference genes). By way of example of housekeeping genes, mentioned may be made of the genes DECR1, HPRT1, PPIB, RPLP0, PPIA, GLYR1, RANBP3, 18S, B2M, TBP, GAPDH and ACTB.

Generally speaking, in the method according to the invention, regardless of the embodiments thereof, the CD74 expression level, preferably the normalized expression, in the biological sample from the subject is compared to a reference value or to the expression of the same gene, preferably the normalized expression, obtained in a reference biological sample.

According to a particular embodiment, the expression of CD74 can indeed be normalized in relation to the expression of one or more housekeeping genes (or reference genes) according to methods known to those skilled in the art. Thus, the expression is normalized using one or more of the following housekeeping genes: DECR1 (chromosomal location: chr8, 90001352-90053633), HPRT1 (chromosomal location: chrX, 134452842-134520513) and PPIB (chromosomal location: chr15:64155812-64163205), RPLP0 (chromosomal location: chr12, 120196699-120201111), PPIA (chromosomal location: chr7, 44795960-44803117), GLYR1 (chromosomal location: chr16, 4803203-4847288), RANBP3 (chromosomal location: chr19, 5916139-5978140), B2M (chromosomal location: chr15, 44711492-44718145), TBP (chromosomal location: chr6, 170554369-170572859), GAPDH (chromosomal location: chr12, 6534517-6538371) and ACTB (chromosomal location: chr14, 5527148-5530601). The chromosomal locations are given according to GRCh38/hg38. Preferably, the expression is normalized using one or more housekeeping genes selected from: DECR1, HPRT1, PPIB, GAPDH and ACTB, and more preferably selected from DECR1, HPRT1 and PPIB.

In such a case, the reference level used is also normalized beforehand, in the same way. The normalization, whether for the reference level or for the level of transcripts of the biological sample to be tested, is carried out before the comparison, particularly before the calculation of a relationship between the level of transcripts of said sample to be tested and the reference level. When a threshold value different from a reference level is used to draw a conclusion, this normalization may be taken into account for the choice of the threshold value.

In the event that the level of CD74 transcripts is normalized relative to the level of transcripts of one or more housekeeping genes, this of course implies that the method according to the invention includes determining the level of transcripts of the housekeeping gene(s) used for the normalization.

According to a particular embodiment, the expression level of the CD74 gene is determined from a biological sample from a subject, and it can be concluded that there is an increased risk of death of said subject when the comparison of the transcript level, particularly mRNA, of said CD74 gene to a predetermined reference threshold value shows that there is a difference to said reference threshold value corresponding to said CD74 gene. In particular, said difference corresponds to a level of transcripts for the biological sample to be tested which is less than that corresponding to the threshold value.

Advantageously, according to a first variant, said threshold value corresponds to a reference expression level of said CD74 gene which is the level of transcripts of said gene obtained from a biological sample from a subject who is not infected with any respiratory virus. According to this variant, the threshold value may also correspond to the average expression level of CD74 transcripts obtained from biological samples originating from a population of subjects who are not infected with any respiratory virus.

According to a second variant, the threshold value corresponds to a reference expression level of the CD74 gene which is the level of transcripts of said gene obtained from a biological sample from a subject who is infected with a respiratory virus but who is known to have survived after infection, particularly in the 28 days following infection. According to this variant, the threshold value can also correspond to the average expression level of CD74 transcripts obtained from biological samples originating from a population of subjects who are infected with a respiratory virus but who are known to have survived after infection, particularly in the 28 days following infection.

Preferably, said difference between the level of transcripts of the CD74 gene determined in the test biological sample and the reference level of said gene (threshold value) corresponds to the fact that the level of transcripts of said gene determined in the test sample is significantly decreased, in particular by at least 10%, at least 20%, at least 30%, at least 40%, at least 50%, at least 60%, at least 70%, at least 80% or at least 90% relative to the reference level, or reference threshold value, of said CD74 gene.

The decrease considered to be relevant for drawing a conclusion will depend on the threshold value in question, and particularly on the reference level used as a reference threshold value, and can be adapted by those skilled in the art. In particular, if the reference threshold value corresponds to the reference level of said CD74 gene which is the level of transcripts of said gene in a subject who is not infected with any respiratory virus, in a population of subjects who are not infected with any respiratory virus, or in a subject or population of subjects who are infected with a respiratory virus but who survived in the 28 days following infection, it may be sufficient for the level of CD74 transcripts determined in the test sample to simply be less than said reference threshold value.

According to the invention, the method makes it possible to conclude that there is an increased risk of death of the subject when under-expression of CD74 is demonstrated in the biological sample to be tested.

“Under-expression” means a significant decrease in the expression level compared to a reference threshold value. Those skilled in the art are able to determine the statistical test to be used to determine this reference threshold value with which the CD74 expression level is to be compared. The exemplary embodiments present one of the possible methods.

According to this particular embodiment, the method may thus comprise the following steps of:
determining the expression level of the CD74 gene by measuring the quantity of at least one CD74 transcript in the biological sample from the subject infected with a respiratory virus,
comparing the quantity of said transcript determined for said biological sample, or a value derived from this quantity, to a reference value predetermined from a reference sample, and
drawing a conclusion regarding the presence of an increased risk of death on the basis of the result of the comparison.

According to this embodiment, the reference sample may for example be a sample originating from a subject or a mixture of samples from a plurality of subjects, said subjects not being infected with a respiratory virus. In this case, the conclusion is drawn that there is an increased risk of death when the comparison of the quantity of transcripts to the reference value demonstrates a significant decrease.

The reference value may particularly correspond to an average value of the CD74 expression level measured from a plurality of samples, each of which originates from different subjects who are not infected with a respiratory virus.

According to a preferred variant of this embodiment, the reference sample is of the same nature as the biological sample to be tested, or at least of a compatible nature to form a reference for determining the CD74 expression level.

A “comparison” or verification of a “difference” between two values or levels of values can be carried out by any known technique. The comparison or verification of a “difference” may involve calculating a relationship or a difference.

In the context of the invention, drawing a conclusion regarding the risk of death of the subject from which the test sample originates may also be carried out by any automated technique performed by a computer or assisted by a computer.

The method as described previously, in all the embodiments thereof, comprises, in addition to the step of measuring the expression of the CD74 gene, a step of measuring, in the biological sample from the patient, the expression of at least one, two, or three additional gene(s) selected from the following genes: TDRD9, IL1R2 and CD177.

The reference values or predetermined threshold value of the expression level of the additional genes may be determined in the same way as the CD74 gene, as described previously.

In particular, for all the embodiments of the present invention, the reference value of the additional genes may advantageously correspond to the level of transcripts, preferably at the mRNA level, of said genes in a subject who is not infected with any respiratory virus or who is infected with a respiratory virus but who survived in the 28 days following infection. The threshold value may also correspond to the average level of transcripts, preferably at the mRNA level, of said genes in a population of subjects who are not infected with any respiratory virus or who are infected with a respiratory virus but who survived in the 28 days following infection.

The chromosomal locations of the additional genes are given in table 1 below:

TABLE 1

| | Chromosomal location | Accession |
|---|---|---|
| Gene (name) | (GRCh38/hg38) | number |
| TDRD9 (Tudor domain | Chromosome 14: 103,928,343- | NM_153046 |
| containing 9) | 104,052,667 | |
| IL1R2 (interleukin 1 | Chromosome 2: 101,991,960- | NM_001261419 |
| receptor type 2) | 102,028,544 | NM_004633 |
| CD177 (CD177 | Chromosome 19: 43,353,686- | NM_020406 |
| molecule) | 43,363,172 | |

According to a preferred variant of this embodiment, the method comprises a step of measuring the expression of the additional gene IL1R2 in the biological sample from the subject. According to this variant, a conclusion can be drawn regarding a risk of death, particularly an increased risk of death, of said subject, when the comparison of the level of mRNA transcripts of the CD74 gene to a predetermined threshold value shows that there is a decrease in the expression level, or under-expression, and when the comparison of the level of mRNA transcripts of the additional gene IL1R2 to a predetermined threshold value shows that there is an increase in the expression level, or over-expression.

According to another variant of this embodiment, the method comprises a step of measuring the expression of the additional gene TDRD9 in the biological sample from the subject. According to this variant, a conclusion can be drawn regarding a risk of death, particularly an increased risk of death, of said subject, when the comparison of the level of mRNA transcripts of the CD74 gene to a predetermined threshold value shows that there is a decrease in the expression level, or under-expression, and when the comparison of the level of mRNA transcripts of the additional gene TDRD9 to a predetermined threshold value shows that there is an increase in the expression level, or over-expression.

According to another variant of this embodiment, the method comprises a step of measuring the expression of the additional gene CD177 in the biological sample from the subject. According to this variant, a conclusion can be drawn regarding a risk of death, particularly an increased risk of death, of said subject, when the comparison of the level of mRNA transcripts of the CD74 gene to a predetermined threshold value shows that there is a decrease in the expression level, or under-expression, and when the comparison of the level of mRNA transcripts of the additional gene CD177 to a predetermined threshold value shows that there is an increase in the expression level, or over-expression.

According to a preferred variant of this embodiment, the method comprises a step of measuring the expression of the additional genes TDRD9 and IL1R2 in the biological sample from the subject. According to this variant, a conclusion can be drawn regarding a risk of death of said subject when the comparison of the level of mRNA transcripts of the CD74 gene to a predetermined threshold value shows that there is a decrease in the expression level, or under-expression, when the comparison of the level of mRNA transcripts of the additional gene TDRD9 to a predetermined threshold value shows that there is an increase in the expression level, or over-expression, and when the comparison of the level of mRNA transcripts of the additional gene IL1R2 to a predetermined threshold value shows that there is an increase in the expression level, or over-expression. According to this variant, the increased expression of TDRD9 and IL1R2 and the decreased expression of CD74 are significantly associated with the death of the subject in the 28 days following infection with the respiratory virus. Still according to this variant, the chances of survival of the subject are approximately 90% when the expression of TDRD9 and IL1R2 are decreased and the expression of CD74 is increased.

According to another variant of this embodiment, the method comprises a step of measuring the expression of the additional genes TDRD9 and CD177 in the biological sample from the subject. According to this variant, a conclusion can be drawn regarding a risk of death, particularly an increased risk of death, of said subject when the comparison of the level of mRNA transcripts of the CD74 gene to a predetermined threshold value shows that there is a decrease in the expression level, or under-expression, when the comparison of the level of mRNA transcripts of the additional gene TDRD9 to a predetermined threshold value shows that there is an increase in the expression level, or over-expression, and when the comparison of the level of mRNA transcripts of the additional gene CD177 to a predetermined threshold value shows that there is an increase in the expression level, or over-expression.

According to another variant of this embodiment, the method comprises a step of measuring the expression of the additional genes CD177 and IL1R2 in the biological sample from the subject. According to this variant, a conclusion can be drawn regarding a risk of death, particularly an increased risk of death, of said subject, when the comparison of the level of mRNA transcripts of the CD74 gene to a predetermined threshold value shows that there is a decrease in the expression level, or under-expression, and when the comparison of the level of mRNA transcripts of the additional genes CD177 and IL1R2 to predetermined threshold values shows that there is an increase in the expression levels, or over-expression of said additional genes.

According to another variant of this embodiment, the method comprises a step of measuring the expression of the additional genes TDRD9, CD177 and IL1R2 in the biological sample from the subject. According to this variant, a conclusion can be drawn regarding an increased risk of death of said subject when the comparison of the level of mRNA transcripts of the CD74 gene to a predetermined threshold value shows that there is a decrease in the expression level, or under-expression, and when the comparison of the level of mRNA transcripts of the additional genes TDRD9, CD177 and IL1R2 to predetermined threshold values shows that there is an increase in the expression level, or over-expression of said additional genes.

According to a particular embodiment, the method may thus comprise the following steps of:
determining the expression level of the genes CD74 and TDRD9 by measuring the quantity of at least one CD74 and TDRD9 transcript in the biological sample from the subject,
comparing the quantity of said transcripts determined for said biological sample, or a value derived from this quantity, to a predetermined reference value obtained from one or more reference samples, and
drawing a conclusion regarding the risk of death on the basis of the result of the comparison.

According to this embodiment, the method makes it possible to conclude that there is a risk of death of the subject when over-expression of TDRD9 and under-expression of CD74 is demonstrated in the biological sample to be tested.

According to a preferred embodiment, the method may thus comprise the following steps of:
determining the expression level of the genes CD74 and IL1R2 by measuring the quantity of at least one transcript of said genes in the biological sample from the subject,
comparing the quantity of said transcripts determined for said biological sample, or a value derived from this quantity, to a reference value for said genes obtained from one or more reference samples, or to a predetermined threshold value, and
drawing a conclusion regarding the risk of death on the basis of the result of the comparison.

According to this embodiment, the method makes it possible to conclude that there is an increased risk of death of the subject when under-expression of CD74 and over-expression of IL1R2 is demonstrated in the biological sample to be tested.

According to another preferred embodiment, the method may thus comprise the following steps of:
determining the expression level of the CD74 gene and of the additional genes IL1R2 and CD177, particularly by measuring the quantity of at least one transcript of said genes in the biological sample from the subject,
comparing the quantity of said transcripts determined for said biological sample, or a value derived from this quantity, to a reference value for each of the genes from one or more reference samples, or to a predetermined threshold value, and
drawing a conclusion regarding the risk of death on the basis of the result of the comparison.

According to this embodiment, the method makes it possible to conclude that there is an increased risk of death of the subject when under-expression of CD74 and over-expression of IL1R2 and CD177 is demonstrated in the biological sample to be tested.

According to another preferred embodiment, the method may thus comprise the following steps of:
determining the expression level of the CD74 gene and of the additional genes IL1R2, CD177 and TDRD9, particularly by measuring the quantity of at least one transcript of said genes in the biological sample from the subject,
comparing the quantity of said transcripts determined for said biological sample, or a value derived from this quantity, to a reference value for each of the genes from one or more reference samples, or to a predetermined threshold value, and
drawing a conclusion regarding the risk of death on the basis of the result of the comparison.

According to this embodiment, the method makes it possible to conclude that there is an increased risk of death of the subject when under-expression of CD74 and over-expression of IL1R2, CD177 and TDRD9 is demonstrated in the biological sample to be tested.

Thus, according to a particular embodiment, the risk of death corresponds to the risk of death of the subject in the 2, 3, 4, 5, 6, 7, 8, 9, 10, 11, 12, 13, 15, 15, 16, 17, 18, 19, 20, 21, 22, 23, 24, 25, 26, 27 or 28 days post-infection of the subject. Preferably, the risk of death corresponds to the risk of death of the subject in the 7 days, 14 days, 21 days, 22 days, 23 days, 24 days, 25 days, 26 days, 27 days or 28 days post-infection of the subject. More preferably, the risk of death corresponds to the risk of death in the 28 days post-infection. The number of days post-infection means the number of days following the subject being identified as positive for an infection with a respiratory virus.

The method as described previously, in all the embodiments thereof, may also comprise, in addition to the step of measuring the expression of the gene(s) described previously, a step of measuring the expression of one or more other additional genes in the biological sample from the subject, said genes being involved in host response and being selected from the panel of genes presented in table 2 below.

TABLE 2

| Gene | Name | Accession number |
|---|---|---|
| ADGRE3 | Adhesion G protein-coupled | NM_032571 |
| | receptor E3 | |
| ARL14EP | Ribosylation factor like | NM_152316 |
| | GTPase 14 effector protein | |
| BPGM | Biphosphoglycerate mutase | NM_199186 |
| | | NM_001293085 |
| | | NM_001724 |
| C3AR1 | Complement C3a receptor 1 | NM_004054 |
| CCNB1IP1 | Cyclin B1 interacting protein 1 | NM_182852 |
| CD274 | CD274 molecule | NM_014143 |
| | | NM_001267706 |
| CD3D | CD3d molecule | NM_000732 |
| | | NM_001040651 |
| CIITA | Class II major histocompatibility | NM000246 |
| | complex transactivator | NM_001286402 |
| | | NM_001286403 |
| CTLA4 | Cytotoxic T-lymphocyte | NM_005214 |
| | associated protein 4 | NM_001037631 |
| CX3CR1 | C-X3-C motif chemokine | NM_001337 |
| | receptor 1 | NM_001171171 |
| | | NM_001171172 |
| | | NM_001171174 |
| GNLY | Granulysin | NM_012483 |
| IFNG | Interferon gamma | NM_000619 |
| IL10 | Interleukin 10 | NM_000572 |
| IL1RN | Interleukin 1 receptor antagonist | NM_173842 |
| IL7R | Interleukin 7 receptor | NM_002185 |
| IP10/ | Interferon gamma induced | NM_001565 |
| CXCL10 | protein 10 | |
| MDC1 | Mediator of DNA damage | NM_014641 |
| | checkpoint 1 | |
| OAS2 | 2′-5′-oligoadenylate synthetase 2 | NM_001032731 |
| | | NM_016817 |
| | | NM_002535 |
| S100A9 | S100 calcium binding protein A9 | NM_002965 |
| TAP2 | Transporter 2, ATP binding | NM_018833 |
| | cassette subfamily B member | NM_001290043 |
| | | NM_000544 |
| TDRD9 | Tudor domain containing 9 | NM_153046 |
| TNF | Tumor necrosis factor | NM_000594 |
| ZAP70 | Zeta chain of T cell receptor | NM207519 |
| | associated protein kinase 70 | |

Another subject of the invention relates to a kit for the in vitro or ex vivo measurement of the expression level of the CD74 gene in a biological sample from a subject, said kit comprising at least one means for determining the CD74 expression level in said biological sample.

The means for determining the expression level of the CD74 gene are known to those skilled in the art and can be specific tools or reagents that make it possible to measure said expression in a biological sample. They may for example be primers or probes.

Of course, the specific or preferred embodiments described in association with the methods according to the invention apply to the kits and uses which are also subject of the invention.

“Primer” or “amplification primer” means a nucleotide fragment which may consist of 5 to 100 nucleotides, preferably 15 to 30 nucleotides, and having hybridization specificity with a target nucleotide sequence under conditions determined for the initiation of enzymatic polymerization, for example in a reaction for the enzymatic amplification of the target nucleotide sequence. Generally, use is made of “primer pairs” consisting of two primers. When it is desired to amplify several different biomarkers (e.g. from different genes), several different pairs of primers are preferably used, each preferentially having the ability to hybridize specifically with a different biomarker.

Those skilled in the art are thus able, from the gene sequence and particularly from the corresponding transcripts, to determine the primers and probes required for amplification, particularly for the amplification of one or more mRNA transcripts of said gene.

“Probe” or “hybridization probe” means a nucleotide fragment typically consisting of 5 to 100 nucleotides, preferably 15 to 90 nucleotides, even more preferably 15 to 35 nucleotides, having hybridization specificity under conditions determined for forming a hybridization complex with a target nucleotide sequence. The probe also comprises a reporter (such as a fluorophore, an enzyme or any other detection system) which will enable the detection of the target nucleotide sequence. In the present invention, the target nucleotide sequence may be a nucleotide sequence contained in a messenger RNA (mRNA) or a nucleotide sequence contained in a complementary DNA (cDNA) obtained by reverse transcription of said mRNA. When it is desired to target several different biomarkers (e.g. from different genes), several different probes are preferably used, each preferentially having the ability to hybridize specifically with a different biomarker.

“Hybridization” means the process during which, under suitable conditions, two nucleotide fragments, for example a hybridization probe and a target nucleotide fragment, having sufficiently complementary sequences, are able to form a double strand with stable and specific hydrogen bonds. A nucleotide fragment which is “able to hybridize” with a polynucleotide is a fragment which can hybridize with said polynucleotide under hybridization conditions, which can be determined in each case in a known way. The hybridization conditions are determined by stringency, i.e. the strictness of the operating conditions. Hybridization is more specific when it is carried out at higher stringency levels. The stringency is defined particularly on the basis of the base composition of a probe/target duplex, and also by the degree of mismatch between two nucleic acids. The stringency can also be based on the reaction parameters, such as the concentration and type of the ionic species present in the hybridization solution, the nature and the concentration of denaturing agents, and/or the hybridization temperature. The stringency of the conditions under which a hybridization reaction must be carried out will chiefly depend on the hybridization probes used. All this information is well known and the suitable conditions can be determined by those skilled in the art.

In general, depending on the length of the hybridization probes used, the temperature for the hybridization reaction is between approximately 20 and 70° C., in particular between 35 and 65° C. in a saline solution at a concentration of approximately 0.5 to 1 M. A step of detecting the hybridization reaction is subsequently carried out.

According to a particular embodiment, the kit further comprises at least one item of threshold value data stored in a computer-readable medium, for example a bar code, and/or used in the form of a code which can be executed by a computer configured to compare the level of CD74 transcripts determined by virtue of the determination means, or an item of data obtained from said level of transcripts of the CD74 gene, to said threshold value.

According to this embodiment, the threshold value may correspond to the level of transcripts, preferably at the mRNA level, of CD74 in a subject who is not infected with any respiratory virus or who is infected with a respiratory virus but who survived in the 28 days following infection. The threshold value may also correspond to the average level of transcripts, preferably at the mRNA level, of CD74 in a population of subjects who are not infected with any respiratory virus or who are infected with a respiratory virus but who survived in the 28 days following infection.

According to another embodiment, the kit further comprises at least one reference level for said marker gene, stored in a computer-readable medium, for example a bar code, and/or used in the form of a code which can be executed by a computer configured to compare the level of CD74 transcripts determined by virtue of the determination means to said reference value, said reference value preferably being the level of CD74 transcripts in a subject who is not infected with a respiratory virus, or in a population of such subjects.

According to another embodiment, the kit further comprises a negative control sample, which is a sample calibrated to contain the quantity of CD74 that corresponds to the quantity or concentration representative of the expression level, measured in a pool of samples from subjects who are known not to be infected with a respiratory virus, and/or a positive control sample, which is a sample calibrated to contain the quantity of CD74 that corresponds to the average quantity measured in a pool of samples from subjects who are known not to have survived, particularly in the 28 days, following infection with a respiratory virus. “Calibrated sample” means a sample prepared so as to comprise the quantity or concentration of CD74 representative of a population of subjects who are infected with a respiratory virus or of subjects who are not infected with a respiratory virus.

According to another embodiment, the kit further comprises a negative control sample, which is a sample calibrated to contain the quantity of CD74 that corresponds to the quantity or concentration representative of the expression level, measured in a pool of samples from subjects who are infected with a respiratory virus and who are known to have survived in the 28 days following infection, and/or a positive control sample, which is a sample calibrated to contain the quantity of CD74 that corresponds to the average quantity measured in a pool of samples from subjects who are known not to have survived, particularly in the 28 days, following infection with a respiratory virus.

In all embodiments thereof, the kit may also comprise at least one additional means for determining the expression level of additional genes, TDRD9, IL1R2 and/or CD177, in said biological sample. According to this embodiment, the kit may comprise, in the same way as defined previously, a threshold value for the expression level of the additional genes and/or of the (positive or negative) control samples of said additional genes. The means for determining the expression level may be specific tools or reagents as defined previously, for example primers or probes.

According to this embodiment, the kit may comprise, in the same way as defined previously, a threshold value for the expression level of the additional genes and/or of the (positive or negative) control samples of said additional genes. The means for determining the expression level may be specific tools or reagents as defined previously, for example primers or probes.

According to a particular embodiment, the kit, in all embodiments thereof, may also comprise, in addition to a means for determining the expression level of CD74 and optionally of the additional genes TDRD9, IL1R2 and/or CD177, at least one other additional means for determining the expression level of one or more genes involved in host response from a biological sample from a subject. The genes involved in host response are as defined in the panel of genes presented in table 2.

The means for determining the expression level may be specific tools or reagents as defined previously, for example primers or probes.

Another subject relates to the use of a kit for in vitro or in vivo measurement, as defined previously, for determining the risk of death of a subject infected with a respiratory virus.

According to a particular embodiment, by additionally measuring the expression level of a panel of genes involved in host response, the kit according to the invention can advantageously be used, in addition to determining the risk of death following infection with a respiratory virus, to determine the immune status of a subject in order to define, particularly, if said subject is immunocompetent or immunosuppressed.

The invention also relates to the in vitro or in vivo determination methods for determining the risk of death of a subject infected with a respiratory virus, such methods being as defined by the present description and further comprising a step of treating said respiratory virus infection.

In particular, treatment can be initiated as soon as a conclusion is drawn regarding the risk of death of said subject infected with a respiratory virus.

The treatment may consist in administering a suitable antiviral drug. As antiviral treatments, mention may particularly be made of Iopinavir®, Ritonavir®, and recombinant interferons, particularly interferon beta, alpha and lambda. Numerous therapeutic treatments for respiratory viruses, particularly those responsible for COVID-19, are currently in the trial phase (Canedo-Marroquín, G. et al., 2020).

According to a particular embodiment, the treatment step consists in administering one or more monoclonal antibodies to said subject. Examples of such antibodies are casirivimab, imdevimab, regdanvimab, tixagevimab or cilgavimab. Preferred treatments may consist of the combination of casirivimab and imdevimab (Ronapreve), or the combination of tixagevimab and cilgavimab.

Another subject relates to a method comprising the following steps:
obtaining a biological sample, preferably a total blood sample, from a subject infected with a respiratory virus, which is SARS-CoV-2,
   bringing said biological sample into contact with specific reagents for the expression products of the target genes CD74, IL1R2, and CD177 and optionally also the expression of the additional target gene TDRD9,
   measuring the expression of said target genes.

The specific reagents for the expression products are selected from amplification primers, hybridization probes or antibodies, and are as defined previously.

According to a particular embodiment, the method may comprise a step of administering a suitable antiviral drug as defined previously, particularly when the expression of the target genes indicate that the subject has an increased risk of death in the 28 days.

Another subject relates to a method for determining whether a patient infected with a respiratory virus has an increased risk of death, comprising the following steps of:
obtaining a biological sample, preferably a total blood sample, from a patient infected with a respiratory virus,
measuring the expression level of the CD74 gene in said biological sample,
comparing the expression level of the CD74 gene to a reference value obtained from patients who are infected with a respiratory virus and who survived,
   wherein, if the expression level of the CD74 gene is less than the reference value, it is determined that the patient has an increased risk of death.

According to a particular embodiment, the method also comprises measuring the expression level of one or more additional genes as defined previously, and comparing the measured levels with respective reference values for each of the additional genes, also obtained from patients who are infected with a respiratory virus and who survived. In this case, the increased risk of death is determined on the basis of the result of the comparison of the expression levels of all the genes measured and as defined previously.

Finally, another subject relates to a method comprising the quantitative measurement, particularly by RT-qPCR, of the mRNA of the genes CD74, IL1R2 and CD177 in a biological blood sample from a subject infected with a respiratory virus such as SARS-CoV-2.

According to a particular embodiment, the method further comprises the quantitative measurement of the mRNA of the TDRD9 gene, particularly by RT-qPCR.

The present invention is illustrated, non-limitingly, using the following examples.

### EXAMPLES

### Materials and Method

### Description of the Two Cohorts of Patients Used

The RICO cohort is a prospective observational clinical study. From this cohort, a sub-cohort of 53 patients (hereinafter referred to as RICO sub-cohort) was formed by recruitment between March and May 2020 within three intensive care units of university hospitals (Hospices Civils de Lyon, Lyon, France). The patients all had a SARS-CoV-2 pulmonary infection confirmed by a RT-PCR test.

In brief, the criteria for inclusion were the following:
(1) male or female ≥18 years old, (2) hospitalization in intensive care unit (ICU) for SARS-CoV-2 pneumopathy, (3) first ICU hospitalization, (4) positive diagnosis of infection with SARS-CoV-2 by PCR or by another approved method in at least one respiratory sample, (5) possibility to take sample in the first 24 h following admission to ICU (D0), and (6) patient or next of kin having been informed about the terms of the study and not being opposed to participation.

The criteria for exclusion were pregnancy, institutionalized patients and where informed consent could not be obtained.

The samples were taken from this sub-cohort on day 0 (in the 48 hours following admission to intensive care), representing 50 patients. The patients had an average age of 64 years [InterQuartile Range 52-70] and exhibited a disparate distribution of men to women (80/20).

REALISM cohort: A subset of 34 RNA samples from healthy volunteers was obtained retrospectively from the REALISM cohort, which is a longitudinal single-centre observational study. The samples for the REALISM study were collected between December 2015 and June 2018. This sub-cohort was used as a control dataset. The healthy volunteers were aged 63.5 years [IQR 52.0-70.0] and exhibited a standard distribution of men to women (50/50).

### Measurements of Immune Markers

The following immune markers are common to the two sub-cohorts: T lymphocyte count, CD4+ T lymphocyte count, CD8+ T lymphocyte count, mHLA-DR (Ab/C), plasma IL-6 and plasma IL-10. Other immune markers were measured only in the RICO sub-cohort.

The plasma concentrations of IL-6, TNF-α, IFN-γ and IL-10 were measured by Simpleplex® technology, using the ELLA instrument according to the manufacturer's instructions. The plasma concentration of IFNα2 were determined by single-molecule array (SIMOA) on an HD-1 analyzer (Quanterix), using a commercial kit for the quantification of IFN-α2. The IFN content was obtained by means of nCounter® analysis technology (NanoString Technologies, Seattle), calculating the median normalized count for the following 6 interferon-stimulated genes (ISG): SIGLEC1, IFI27, IFI44L, IFIT1, ISG15 and RSAD2. Immunophenotyping of the T lymphocyte sub-population was performed using an automated volumetric flow cytometer from Beckman Coulter (Aquios CL). The HLA-DR expression by monocytes was achieved using antibodies from Beckman-Coulter and BD Biosciences (San Jose, USA). The HLA-DR expression by monocytes was determined using the Anti-HLA-DR/Anti-Monocyte Quantibrite test (BD Biosciences, San Jose, USA). The total number of antibodies bound per cell, expressed as Ab/C, was quantified using calibration with a standard curve determined with BD Quantibrite phycoerythrin (PE) beads (BD Biosciences).

Blood samples were collected in PAXgene® tubes (ref. 762165, PreAnalytiX GmbH Hombrechtikon Switzerland) at D0 (48 h post-admission to ICU).

The total RNA was extracted from the PAXgene™ tubes using the Maxwell® 16 LEV simplyRNA Blood kit (Promega), according to the manufacturer's instructions. The quantity of RNA was determined using a NanoVue (Biochrom).

Next, 200 ng of total RNA were tested using a FilmArray® pouch optimized for detecting a panel of genes involved in host response, including CD74, by nested PCR. The pouches were analyzed on the FilmArray® Torch instrument (BioFire®, USA) which performs the steps of nucleic acid extraction, reverse transcription and qPCR automatically.

The normalized expression values for the markers (in relation to the reference genes DECR1, HPRT1 and PPIB) were calculated and used for the analyses.

### Statistical Analysis

The qualitative data was reported in numerical form or as concentrations, and the quantitative data was reported in the form of a median [IQR range]. The clinical or immunological characteristics of the volunteers from the RICO sub-cohort and of the healthy volunteers (REALISM cohort) were compared using the Mann-Whitney-Wilcoxon nonparametric test for continuous variables or Fisher's exact test or the chi-squared test for the categorical variables. Significance was set at 5% for the bilateral tests. The statistical analyses were carried out using the R software, version 4.0.4. The data were centered to perform unsupervised principle component analysis.

The correlation between the gene expression markers and the immunological parameters was evaluated using Spearman's ρ correlation scores. The whole gene panel of the FilmArray® pouch was used, without applying any characteristic selection, in order to build five models for predicting survival at 28 days, namely: Elastic net, LASSO, Ridge regression, random forest (rf) and Partial Least Squares Discriminant Analysis (PLS) using the CARET package (version 6.0-88). The hyperparameters were set and the models were adjusted using cross-validation (k-fold=3, number of repetitions=10) in the RICO sub-cohort.

In summary, among the 5 machine learning algorithms evaluated, Elastic net (α=0.1, λ=0.89898) had the best performance, with an area under the ROC curve (AUC) of 0.675 [confidence interval], LASSO (α=1, λ=0.112) was classed second with an AUC of 0.671, then PLS (ncomp=1) with an AUC of 0.668, the Ridge regression model (α=0, λ=1) was fourth with an AUC of 0.650, and finally Random Forest (mtry=5) was last in terms of performance, with an AUC of 0.63.

The biomarkers of interest were identified by the importance of the variables provided by each model in the incidence of death at 28 days. These biomarkers identified by the machine learning algorithms are the same as those that result significantly from the univariate analysis of association with mortality at 28 days. The patients were stratified into 2 groups on the basis of the Youden threshold, determined for each biomarker, for the prediction of mortality at 28 days in the total population of patients on admission, and the non-adjusted cumulative incidences of death were obtained using the Kaplan-Meier estimations.

### Ethics

The protocol for the REALISM study was approved by the IRB (Comité de Protection des Personnes Sud-Est II, approval number 2015-42-2). This clinical study was registered at clinicaltrials.gov (NCT02638779). Informed consent was obtained for each patient and/or from their next of kin; the patients or legal representatives were informed of the study of their right to refuse to participate.

The protocol for the RICO study was approved by the ethics committee (Comité de Protection des Personnes Ile de France 1—IRB/IORG number: IORG0009918) under agreement number 2020-A01079-30. This clinical study was registered at ClinicalTrials.gov (NCT04392401). The committee waived the need for written informed consent since it was an observational study with a low risk for the patients and since no specific procedure other than taking a routine blood sample was required.

The two cohorts are compliant with the Declaration of Helsinki, with the principles of good clinical practice and with French law on personal data protection.

### Results

### Clinical Characteristics on Admission to Intensive Care Unit

The characteristics of the patients from the RICO sub-cohort and of the healthy volunteers are presented in table 3 below. The patients were hospitalized in the 3 hospitals in Lyon over the period which coincided with the first wave of COVID-19 cases in France (March-May 2020).

In brief, and as mentioned previously, 80% of the patients were men. The patients were admitted to hospital a median of 8 days after the first symptoms presented [IQR 5-10], without any significant difference between survivors and non-survivors at 28 days. 44% of the patients had at least one comorbidity, with diabetes being the most frequent (13/22 patients). The patients had a median body mass index (BMI) (kg/m3) of 29.33 [IQR, 26.58-31.54], without any difference between survivors and non-survivors.

In terms of disease severity, the patients had a high PaO₂/FiO₂ [median: 134; IQR: 90.5-191] and a SAPS II score [median: 33.5; IQR: 24.5-43.25]. Among the 50 patients, 11 (22%) died by the 28th day.

It should be noted that the non-survivors had a significantly higher SAPS II score [median: 39; IQR: 34-53.5] compared to the survivors [median: 32; IQR: 22-39.5] (p value=0.023).

Overall, the survivors spent a median duration of 30 days [IQR: 13.25-62.5] in hospital, including 10 days [IQR: 3-40] in intensive care. Half of this RICO sub-cohort developed secondary infections (52%), without a difference between survivors and non-survivors.

TABLE 3

| | | | | Did not | |
|---|---|---|---|---|---|
| | | | Survived at 28 | survive at 28 days | p |
| | | Patients (n = 50) | days (n = 39) | (n = 11) | value |
| Demographics | | | | | |
| Age | | 64 [52-70] | 63 [51-69.5] | 67 [60-78] | 0.096 |
| Gender, male | | 40 (80%) | 30 (76.92%) | 10 (90.91%) | 0.424 |
| BMI [kg/m³] | | 29.33 | 29.41 | 28.28 | 0.292 |
| | | [26.58-31.54] | [26.86-32.2] | [24.45-29.91] | |
| BMI >30 kg/m³ | | 19 (38%) | 16 (41.03%) | 3 (27.27%) | 0.498 |
| Comorbidities | | | | | |
| Diabetes | None | 37 (74%) | 30 (76.92%) | 7 (63.64%) | 0.197 |
| mellitus | With | 3 (6%) | 1 (2.56%) | 2 (18.18%) | |
| | deterioration | | | | |
| | Without | 10 (20%) | 8 (20.51%) | 2 (18.18%) | |
| | organic | | | | |
| | deterioration | | | | |
| Comorbidities | >=1 | 22 (44%) | 16 (41.03%) | 6 (54.55%) | 0.503 |
| | 0 | 28 (56%) | 23 (58.97%) | 5 (45.45%) | |

| | | | | |
|---|---|---|---|---|
| Charlson score | 0 [0-2] | 0 [0-1] | 1 [0-2] | 0.243 |
| Symptoms on admission | | | | |
| Time from first symptoms | 8 [5-10] | 8 [5.5-10.5] | 6 [4-7] | 0.079 |
| (days) | | | | |
| Severity score | | | | |
| SOFA Score | 4 [2-8] | 3 [2-8] | 7 [4.5-8] | 0.068 |
| | 33.5 | 32 | 39 | 0.023 |
| SAPS II Score | [24.5-43.25] | [22-39.5] | [34-53.5] | |
| PaO₂/FiO₂ ratio on | 134^{a} | 152.5^{b} | 120 | 0.429 |
| admission | [90.5-191] | [88.5-204.75] | [101-135.5] | |
| Antiviral therapy | | | | |
| Hydroxychloroquine | 22 (62.86%) | 17 (60.71%) | 5 (71.43%) | 0.689 |
| Lopinavir/ritonavir | 5 (14.29%) | 5 (17.86%) | 0 (0%) | 0.559 |
| Lopinavir/ritonavir + Interferon β | 5 (14.29%) | 4 (14.29%) | 1 (14.29%) | 1 |
| Remdesivir | 1 (2.86%) | 1 (3.57%) | 0 (0%) | 1 |
| Organ support | | | | |
| Mechanical ventilation on | 30 (60%) | 20 (51.28%) | 10 (90.91%) | 0.033 |
| admission | | | | |
| Invasive mechanical | 19 (38%) | 13 (33.33%) | 6 (54.55%) | 0.293 |
| ventilation | | | | |
| Vasoactive drugs | 19 (38%) | 13 (33.33%) | 6 (54.55%) | 0.293 |
| Renal replacement therapy | 10 (20%) | 7 (17.95%) | 3 (27.27%) | 0.671 |
| Follow-up care | | | | |
| Days in ICU | 9 [4-29.75] | 10 [3-40] | 8 [6-19] | 0.716 |
| Days in hospital | 21 [13-56] | 30 [13.25-62.5] | 15 [7.5-20] | 0.007 |
| Mortality at 28 days | 11 (22%) | 0 (0%) | 11 (100%) | <0.001 |
| Mortality at 90 days | 14 (28.57%) | 3 (7.89%) | 11 (100%) | <0.001 |
| Infections acquired in ICU | 26 (52%) | 19 (48.72%) | 7 (63.64%) | 0.501 |

The medians and interquartile ranges [Q1-Q3] are indicated for continuous variables where the numbers and percentages are presented for categorical variables. The patients from the COVID-19 study were separated into two groups according to their survival status 28 days after admission. The sequential organ failure (SOFA) and simplified acute physiology II (SAPS II) scores were calculated during the first 24 hours after admission. The data were compared using the Mann-Whitney nonparametric test for continuous variables and/or Fisher's exact test for categorical variables.^{a,b} Three patients did not have a PaO₂/FiO₂, giving rise to 47 measurements (out of 50) in the column for all patients and 36 measurements (out of 39) in the survivors at 28 days.

### Association Between Biomarker Expression and Clinical Status of Patients

The relationship between the transcriptomic markers from the FilmArray® pouch and the different clinical results was analyzed.

Firstly, the association with the severity of the patients on admission, as reflected by the clinical scores, was studied in order to confirm the potential of the biomarkers. It is particularly clear that CD74 is negatively correlated with the SAPS II score (ρ=−0.30).

On the other hand, increased expression of TDRD9, CD177, and IL1R2 was positively correlated with the SAPS II score (TDRD9: ρ=0.46, CD177: ρ=0.5, and IL1R2: ρ=0.47).

### Association Between Expression of Biomarkers and Mortality of Patients at 28 Days

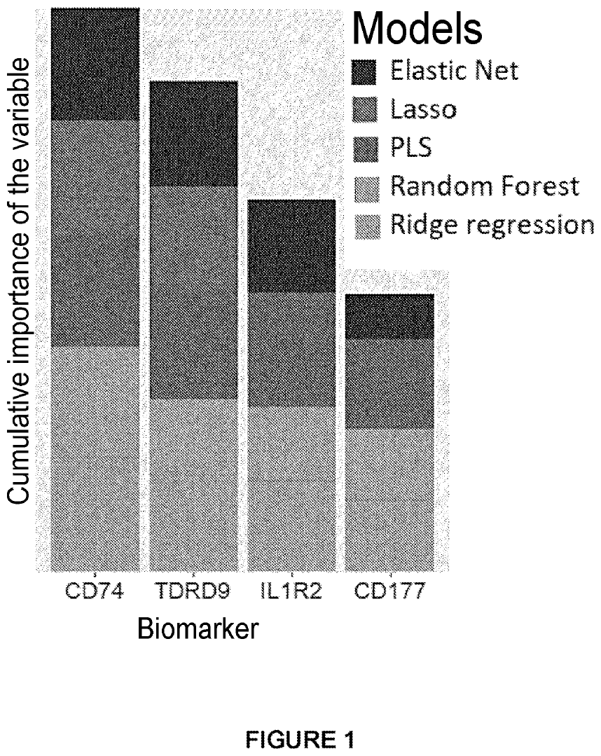

Using 5 different machine learning approaches, 4 genes associated with the prediction of mortality at 28 days were identified. These are the genes CD74, TDRD9, IL1R2 and CD177 (FIG. 1).

These four markers are expressed differentially in a univariate analysis between surviving and non-surviving patients (table 4).

TABLE 4

| | OR_{IQR} [95% CI] | IQR | p value |
|---|---|---|---|
| TDRD9 | 4.221 | 2.01 | 0.007 |
| | [1.591-13.621] | | |
| CD177 | 4.085 | 3.58 | 0.027 |
| | [1.315-16.548] | | |
| CD74 | 0.202 | 1.14 | 0.007 |
| | [0.054-0.569] | | |
| IL1R2 | 3.548 | 2.28 | 0.010 |
| | [1.448-10.424] | | |
| mHLA-DR | 1.072 | 7335 | 0.868 |
| [Ab/C] | [0.435-2.402] | | |
| CD3 T cells | 0.26 | 362 | 0.050 |
| [cells/μL] | [0.054-0.816] | | |
| Plasma IL-6 | 1.338 | 142 | 0.187 |
| [pg/μL] | [1.034-2.3] | | |
| Plasma IL-10 | 1.116 | 16 | 0.221 |
| [pg/μL] | [0.945-1.407] | | |

The association between the survival status at 28 days and transcriptomic markers or conventional immune parameters was made by implementing univariate logistic regression models, and the odds ratios (OR) of the interquartile range (IQR) were extracted. In order to enable comparison between models, the odds ratios (OR) calculated for each immune parameter and transcriptomic parameter were normalized to an increment of the first to third quartile. The p≤0.05 values are highlighted in bold.

More specifically, CD74 was significantly down-regulated in non-surviving patients compared to surviving patients.

Regarding the expression level of additional genes, IL1R2 and CD177 were significantly up-regulated in non-surviving patients compared to surviving patients, while TDRD9 was significantly up-regulated in non-surviving patients compared to the survivors, and to a greater extent than in the healthy volunteers.

In contrast, the immunological parameters measured on admission (monocyte HLA-DR, plasma IL-6 and IL-10) were not significantly associated with the survival status of patients at 28 days, thereby fully demonstrating the predictive potential of CD74 and of the additional markers according to the invention.

On the basis of the observation that age and invasive mechanical ventilation are associated with severity in patients suffering from COVID-19 (cdc.gov/coronavirus/2019-nCoV), these parameters were used as confounding factors in a multivariate analysis [3a]. We observed that the biomarker CD74 and the additional biomarkers TDRD9, IL1R2 and CD177 remained significantly associated with survival and had an interquartile odds ratio (OR) of 5.35 for TDRD9, 0.22 for CD74, 3.26 for IL1R2 and 5.22 for CD177 (results not shown).

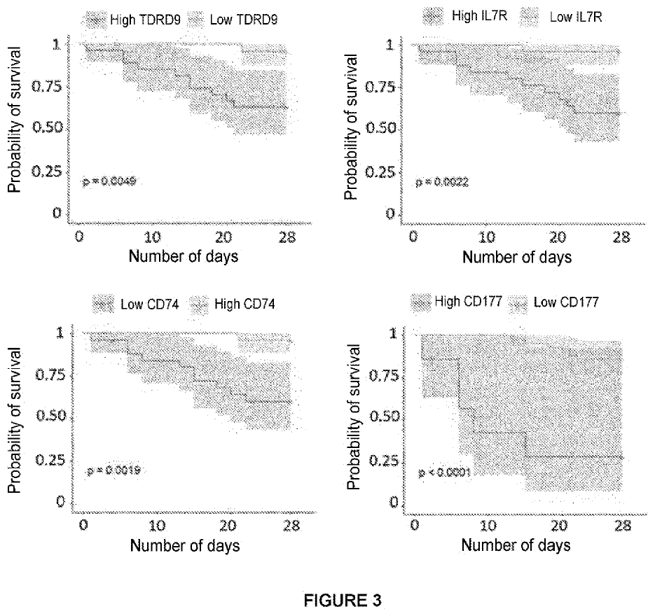

By means of Kaplan-Meier curves with the expression for each gene, using the Youden threshold (for prediction of survival of the patients at 28 days following infection) to distinguish between low/high expression, it was identified that the following expression levels: TDRD9^{high} CD74^{low}, CD177^{high} and IL1R2^{high} were significantly associated with death over a period of 28 days, while the chances of survival were approximately 90% for patients having the following expression levels: TDRD9^{low}, CD74^{high}, CD177^{low} and IL1R2^{low} (FIG. 3). These results thus once again demonstrate the predictive value of the biomarkers according to the invention in determining the risk of death of patients infected with a respiratory virus of the SARS-Cov-2 type, particularly the risk of death at 28 days.

The results obtained above with the CD74 gene and the additional genes were confirmed using the RICO cohort, more specifically in a second sub-cohort of 306 patients recruited between August 2020 and August 2021 within the three intensive care units of the university hospitals (Hospices Civils de Lyon, Lyon, France).

### BIBLIOGRAPHICAL REFERENCES

C. Huang, et al., “Clinical features of patients infected with 2019 novel coronavirus in Wuhan, China” Lancet, 2020, 395, 497, https://doi.org/10.1016/S0140-6736(20)30183-5;
E. Z. Ong, et al., “A Dynamic Immune Response Shapes COVID-19 Progression” Cell Host Microbe, 2020, 27 (6), 879, https://doi.org/10.1016/j.chom.2020.03.021;
F. Venet et al., “Longitudinal assessment of IFN-I activity and immune profile in critically ill COVID-19 patients with acute respiratory distress syndrome”; Crit Care, 2021, 25 (1), 140, https://doi.org/10.1186/s13054-021-03558-w;
D. Mathew, et al., “Deep immune profiling of COVID-19 patients reveals distinct immunotypes with therapeutic implications”; Science, 2020, 369 (6508), https://doi.org/10.1126/science.abc8511;
A. G. Laing et al., “A dynamic COVID-19 immune signature includes associations with poor prognosis”, Nat Med, 2020, 26 (10), 1623, https://doi.org/10.1038/s41591-020-1038-6.
M. S. Abers, et al., “An immune-based biomarker signature is associated with mortality in COVID-19 patients” JCI Insight, 2021, 6 (1):e144455, https://doi.org/10.1172/jci.insight.144455;
Guardela et al., “50-gene risk profiles in peripheral blood predict COVID-19 outcomes: A retrospective, multicenter cohort study” EBioMedecine, 2021, https://doi.org/10.1016/j.ebiom.2021.103439;
Y. Levy et al., “CD177, a specific marker of neutrophil activation, is associated with coronavirus disease 2019 severity and death” Iscience, 2021, 24(7):102711 DOI: 10.1016/j.isci.2021.102711;
G. Canedo-Marroquín et al., “SARS-CoV-2: Immune Response Elicited by Infection and Development of Vaccines and Treatments” Front. Immunol, 2020, 11:569760; DOI 10.3389/fimmu.2020.569760.

What is claimed is:

1. An in vitro or ex vivo method for determining the risk of death of a subject infected with a respiratory virus, comprising a step of measuring the expression level of the CD74 gene in a biological sample from said subject.

2. The method as claimed in claim 1, wherein the respiratory virus is SARS-CoV-2 or a variant thereof.

3. The method as claimed in claim 1 further comprising the following steps of:
comparing the expression level of the CD74 gene for said biological sample, or a value derived from this quantity, to a predetermined threshold value, and
drawing a conclusion regarding the risk of death on the basis of the result of the comparison.

4. The method as claimed in claim 3, wherein the reference threshold value corresponds to the average expression level of the gene obtained from biological samples originating from a population of subjects who are not infected with any respiratory virus, or to the average expression level of said gene obtained from biological samples originating from a population of subjects who are infected with a respiratory virus and who are known to have survived following infection, particularly in the 28 days following infection.

5. The method as claimed in claim 1 further comprising measuring the expression of at least one other additional gene selected from IL1R2, TDRD9 and/or CD177 in the biological sample from said subject.

6. The method as claimed in claim 5 further comprising measuring the expression of the two additional genes IL1R2 and CD177 in the biological sample from the subject, and comparing the expression levels to predetermined threshold values for each of said additional genes.

7. The method as claimed in claim 6, further comprising determining that there is an increased risk of death of said subject when the comparison of the expression level of the CD74 gene to the predetermined threshold value shows that there is under-expression, and when the comparison of the expression level of the additional genes IL1R2 and CD177 to predetermined threshold values shows that there is over-expression.

8. The method as claimed in claim 1, wherein the biological sample is a blood sample.

9. The method as claimed in claim 1, wherein the expression level is measured at the messenger RNA (mRNA) level.

10. The method as claimed in claim 1, wherein the expression is measured by RT-PCR by sequencing or by hybridization.

11. The method as claimed in claim 9, wherein the expression is normalized in relation to the expression of one or more housekeeping genes.

12. The method as claimed in claim 1 further comprising measuring the expression of one or more other additional genes in the biological sample from the subject, said genes being involved in host response and being selected from ADGRE3, ARL14EP, BPGM, C3AR1, CCNB1IP1, CD274, CD3D, CIITA, CTLA4, CX3CR1, GNLY, IFNG, IL10, IL1RN, IL7R, IP10/CXCL10, MDC1, OAS2, S100A9 and combinations thereof in the biological sample from the subject.

13. A kit for the in vitro or ex vivo measurement of CD74 expression level in a biological sample, comprising means for determining the CD74 expression level in said sample, said means being primers or probes, said kit being used to determine the risk of death of a subject infected with a respiratory virus.

14. The kit for use thereof as claimed in claim 13 comprising a negative control sample comprising the quantity of CD74 that corresponds to the quantity or concentration representative of the expression level, measured in a pool of samples from subjects who are not infected with a respiratory virus, and/or a positive control sample comprising the quantity of CD74 that corresponds to the average quantity measured in a pool of samples from subjects who did not survive following infection with a respiratory virus.

15. The kit for use thereof as claimed in claim 13 further comprising means for determining the expression level of at least one other additional gene selected from TDRD9, IL1R2 and CD177.

16. The kit for use thereof as claimed in claim 13 further comprising means for determining the expression level of one or more additional genes involved in host response, as defined in claim 10.

17. The kit for use thereof as claimed in claim 13, wherein the risk of death corresponds to the risk of death in the 28 days post-infection.
